(19) United States
(12) Patent Application Publication
(10) Pub. No.: US 2026/0334473 A1
(43) Pub. Date: Oct. 29, 2026

(54) ANTIMICROBIAL COMPOSITIONS AND METHODS

(71) Applicant: EVOQ NANO, INC., Salt Lake City, UT (US)

(72) Inventor: William H. Niedermeyer, West Jordan, UT (US)

(21) Appl. No.: 19/660,008

(22) Filed: Apr. 27, 2026

Related U.S. Application Data
(63) Continuation-in-part of application No. 17/878,652, filed on Aug. 1, 2022, now Pat. No. 12,610,952.
Application No. 17/878,652 is a continuation-in-part of application No. 16/012,508, filed on Jun. 19, 2018, now abandoned.
Application No. 16/012,508 is a division of application No. 14/861,243, filed on Sep. 22, 2015, now abandoned.
(60) Provisional application No. 62/054,152, filed on Sep. 23, 2014.
(60) Provisional application No. 62/054,154, filed on Sep. 23, 2014.

(51) Int. Cl. A01N 59/16; A61K 33/24; A61K 33/242; A61K 33/243; A61K 33/244

(52) U.S. Cl. CPC A01N 59/16; A61K 33/24; A61K 33/242; A61K 33/243; A61K 33/244

(57) ABSTRACT

Antimicrobial compositions for killing or deactivating microbes, such as viruses, bacteria, or fungi, include metal nanoparticles, a carrier, and a plurality of metal nanoparticles. The nanoparticles can be selected to have a particle size and particle size distribution to selectively and preferentially kill one of a virus, a bacterium, or a fungus. Antibacterial compositions can include nanoparticles having a particle size of 3-14 nm, 5-13 nm, 7-12 nm, or 8-10 nm. Antimicrobial compositions can include nonionic, ground state silver nanoparticles made by laser ablation at a concentration of about 0.25 ppm to about 100 ppm, or about 0.5 ppm to about 50 ppm, or about 1 ppm to about 25 ppm.

### CROSS-REFERENCE TO RELATED APPLICATIONS

This application is a continuation-in-part of U.S. patent application Ser. No. 17/878,652, filed Aug. 1, 2022, which is a continuation-in-part of U.S. patent application Ser. No. 16/012,508, filed Jun. 19, 2018, which is a division of U.S. patent application Ser. No. 14/861,243, filed Sep. 22, 2015, which claims the benefit of U.S. Provisional Patent Application No. 62/054,152, filed Sep. 23, 2014, and U.S. Provisional Patent Application No. 62/054,154, filed Sep. 23, 2014. Each of the foregoing is incorporated herein by reference in its entirety.

### BACKGROUND

### 1. Field of the Invention

Disclosed herein are antimicrobial compositions and methods for killing microbes using such compositions.

### 2. Relevant Technology

Diseases old and new caused by microbes such as bacteria, viruses, and fungi continue to plague humans, animals, and plants. In addition to infecting humans, sometimes as epidemics or pandemics, microbial diseases that affect animals and plants raised for food have and can continue to devastate food supplies. Food products are also prone to spoilage as a result of microbes, particularly bacteria and fungi.

Although modern science has yielded many new antimicrobial compositions, some, such as antibiotics, are not always effective because microbes can build up tolerance or immunity to such compositions. In addition, apart from natural immunity and acquired immunity from vaccinations, there are no compositions that can reliably target and selectively destroy viruses.

Besides specific compositions and drugs, there are non-specific ways to combat microbes and microbial diseases, such as burning, harsh chemicals, and partial or complete removal of diseased human, animal, or plant tissues. Because such measures are non-specific and often kill or severely affect the organism being treated, they are usually used as a last resort.

Among the deadliest human diseases are anthrax, caused by Bacillus Anthracis; ebola virsus disease; hemorrhagic fever, caused by Marburg virus; hantavirus; rabies; smallpox; Crimean-Congo hemorrhagic, fever caused by a tick-borne virus; avian influenza (bird flu); severe acute respiratory syndrome (SARS), caused by coronavirus; malaria, caused by mosquitos infected with Plasmodium parasites; typhoid fever caused by Salmonella typhi bacterium; cholera, an acute intestinal infection caused by Vibrio cholera bacterium; yellow fever, a hemorrhagic fever transmitted by infected mosquitoes; acquired immune deficiency syndrome (AIDS); bubonic plague, caused by Yersinia pestis bacterium; and wound and flesh infections caused by many different bacteria, particularly those which are antibiotic-resistant.

There are also many plant diseases of many types that could threaten the world's food supply. An example of a potentially devastating plant disease is citrus greening disease, which affects citrus trees and for which there is no cure. Citrus greening disease is caused by motile bacteria, Candidatus Liberibacter spp. and transmitted by insects (e.g., various types of psyllids). In the heyday of Florida citrus, around 1970, the number of acres with orange, grapefruit, and specialty fruit orchards surpassed 900,000. Today, it is reportedly slightly more than 500,000 acres as a result of citrus greening disease.

Botulism from consuming spoiled food is a relatively rare but serious and potentially fatal paralytic illness caused by a nerve toxin that is produced by the bacterium Clostridium botulinum.

Among the deadliest viral diseases is Ebola disease, with a mortality rate that is reportedly as high as 90%. Ebola virus disease (EVD), Ebola hemorrhagic fever (EHF), or simply Ebola is a disease of humans and other primates caused by an Ebola virus. Symptoms of the disease can start two days to three weeks after contracting the virus, with a fever, sore throat, muscle pain and headaches. Typically, vomiting, diarrhea and rash follow, along with decreased functioning of the liver and kidneys. Around this time, affected people may begin to bleed both within the body and externally.

EVD is caused by four of five viruses classified in the genus Ebolavirus, family Filoviridae, order Mononegavirales. The four disease-causing viruses are Bundibugyo virus (BDBV), Sudan virus (SUDV), Tai Forest virus (TAFV), and one called simply, Ebola virus (EBOV, formerly Zaire Ebola virus). Ebola virus is the sole member of the Zaire ebolavirus species, and the most dangerous of the known Ebola disease-causing viruses, as well as being responsible for the largest number of outbreaks. The fifth virus, Reston virus (RESTV), is not thought to be disease-causing in humans. The five Ebola viruses are closely related to Marburg viruses.

The overuse of antibiotics has contributed, in some cases, to antibiotic resistant bacteria and other treatment resistant microbes. There is concern that an increase in antibiotic resistance may lead to microbes that are untreatable with conventional technologies.

There are efforts to utilize silver ion-releasing colloidal silver as an antimicrobial agent. However, antimicrobial resistance has now been discovered for colloidal silver (i.e., silver nanoparticles manufactured by conventional chemical reduction processes, typically with some form of capping agent, which are known to release silver ions) and ionic silver. McNeilly et al., “Emerging Concern for Silver Nanoparticle Resistance in Acinetobacter baumannii and Other Bacteria,” Front. Microbiol., 16 Apr. 2021, discuss the emergence of several antibiotic-resistant bacteria, including Acinetobacter baumannii, Enterococcus faecium, Staphylococcus aureus, Klebsiella pneumoniae, Acinetobacter baumannii, Pseudomonas aeruginosa, and Enterobacter spp. Of these, A. baumannii was of particular concern and was found to also have developed resistance to colloidal silver nanoparticles, as were E. Coli, Enterobacter cloacae, S. typhimurium, B. subtilis, S. aureus. P. aeruginosa, K. pneumoniae, Serratia marcescens, and Acinetobacter spp.

Silver, “Bacterial silver resistance: molecular biology and uses and misuses of silver compounds,” FEMS Microbiology Reviews, Volume 27, Issue 2-3, June 2003, Pages 341-35, discusses silver-resistant Salmonella, and Escherichia coli. Elkrewi, et al., “Cryptic silver resistance is prevalent and readily activated in certain Gram-negative pathogens,” J. Antimicrob. Chemother., 2017 Nov. 1; 72(11):3043-3046 discloses colloidal silver nanoparticle resistance by gram negative pathogens, such as Enterobacter spp., Klebsiella spp. Escherichia coli, Pseudomonas aeruginosa, Acinetobacter spp., Citrobacter spp., and Proteus spp. Hosney, “The increasing threat of silver-resistance in clinical isolates from wounds and burns,” Infect Drug Resist. 2019; 12: 1985-2001 discusses colloidal silver-resistant Klebsiella pneumoniae, Staphylococcus aureus, Escherichia coli, Enterobacter cloacae, Pseudomonas aeruginosa, and Acinetobacter baumannii. Percival, et al., “Bacterial resistance to silver in wound care, J. Hospital Infection, Vol. 60, Issue 1, May 2005, pp. 1-7, discusses the fear and possibility of colloidal silver-resistant microbes in wounds. Kȩdziora, et al., “Consequences Of Long-Term Bacteria's Exposure To Silver Nanoformulations With Different PhysicoChemical Properties,” Intl. J. of Nanomedicine, 2020:15 199-213, discusses colloidal silver-resistant gram positive and gram negative bacteria.

An article entitled “Are Silver Nanoparticles a Silver Bullet Against Microbes?” Jul. 13, 2021, https:/news.engineering.pitt.edu/are-silver-nanoparticles-a-silver-bullet-against-microbes/, discusses colloidal silver nanoparticle resistant E. coli., stating: “In the beginning, bacteria could only survive at low concentrations of silver nanoparticles, but as the experiment continued, we found that they could survive at higher doses . . . . Interestingly, we found that bacteria developed resistance to the silver nanoparticles but not their released silver ions alone.” The group sequenced the genome of the E. coli that had been exposed to silver nanoparticles and found a mutation in a gene that corresponds to an efflux pump that pushes heavy metal ions out of the cell. “It is possible that some form of silver is getting into the cell, and when it arrives, the cell mutates to quickly pump it out . . . . More work is needed to determine if researchers can perhaps overcome this mechanism of resistance through particle design.”

Silver nanoparticles made by conventional chemical synthesis methods have external bond angles and edges where silver ions can be released, even though the bulk nanoparticles are ground state. Using metal nanoparticles that release ions provides a source of unwanted metal ions, such as silver ions, which may be toxic to human and animal tissues under excess exposure. Indeed, the problems associated with the release of silver ions are well-known. Prabhu et al., “Silver nanoparticles: mechanism of antimicrobial action, synthesis, medical applications, and toxicity effects,” International Nano Letters 2012, 2:32 (http:/iwww.inl-joumal.com/content/2/1/32, states: “Hence, this current review concludes with a hope and prayer that there would be mechanisms devised to nullify any toxicity caused by nanosilver to humans and the environment so that the unique properties of this substance can be put to great use for human betterment without any controversies.”

Accordingly, there has been and remains a need to find compositions that can reliably target and preferentially kill or deactivate disease-causing microbes without killing or causing undue harm to the organism being treated (e.g., human, animal, or plant).

### SUMMARY

Disclosed herein are antimicrobial compositions and methods for killing or deactivating a wide variety of harmful microbes, such as viruses, bacteria, and fungi, which can infect humans, animals, plants, or food supplies. Also disclosed are methods for making antimicrobial compositions. Unexpectedly, it has now been found that by selecting metal nanoparticles of a particular size and particle size distribution it is possible to target and preferentially kill or deactivate a specific type of microbe. In addition, silver nanoparticles made by laser ablation so as to be nonionic, ground state, and without external edges or bond angles that cause release of metal ions provide unexpectedly superior antimicrobial activity compared to colloidal silver (i.e., silver nanoparticles manufactured by conventional chemical reduction processes, typically with some form of capping agent, which are known to release silver ions).

In some embodiments, an antimicrobial composition may comprise (1) a carrier and (2) a plurality of metal nanoparticles having a particle size and a particle size distribution selected so as to selectively and preferentially kill one of a virus, bacterium, or fungus.

In some embodiments, anti-viral compositions can include metal nanoparticles having a particle size of about 8 nm or less, or about 1 nm to about 7 nm, or about 2 nm to about 6.5 nm, or about 3 nm to about 6 nm. Within these size ranges it is possible to select “designer anti-viral particles” of specific size that are particularly effective in targeting a specific virus.

In some embodiments, an anti-viral composition may comprise a plurality of non-ionic metal nanoparticles having a particle size in a range of about 0.22 nm to about 2 nm, or about 0.22 nm to about 1.5 nm, or about 0.24 nm to about 1 nm, or about 0.27 nm to about 0.7 nm, or about 0.3 nm to about 0.5 nm, or about 0.35 nm to about 0.45 nm. For example, such embodiments may be useful for targeting and preferentially deactivating Ebola viruses.

In some embodiments, anti-viral compositions can include metal nanoparticles having a particle size of about 2 nm to about 10 nm, or about 4 nm to about 10 nm, or about 6 nm to about 10 nm. For example, such embodiments with particles that are 6 nm, 10 nm, or both may be useful for targeting and preferentially deactivating Ebola viruses by associating with, binding to, and/or disrupting the 3CSY glycoprotein found on the surface of Ebola viruses, thereby preventing binding of the virus to cell surface receptors.

In some embodiments, anti-bacterial compositions can include metal nanoparticles having a particle size of about 3 nm to about 14 nm, or about 5 nm to about 13 nm, or about 7 nm to about 12 nm, or about 8 nm to about 10 nm. Within these size ranges it is possible to select “designer anti-bacterial particles” of specific size that are particularly effective in targeting a specific bacterium.

In some embodiments, anti-fungal compositions can include metal nanoparticles having a particle size of about 9 nm to about 20 nm, or about 10 nm to about 18 nm, or about 11 nm to about 16 nm, or about 12 nm to about 15 nm. Within these size ranges it is possible to select “designer anti-fungal particles” of specific size that are particularly effective in targeting a specific fungus.

In some embodiments, metal nanoparticles can comprise spherical-shaped metal nanoparticles having a mean diameter and a particle size distribution wherein at least 99% of the metal nanoparticles have a particle size within 30% of the mean diameter, or within 20% of the mean diameter, or within 10% of the mean diameter and/or wherein at least 99% of the spherical-shaped nanoparticles have a diameter within +3 nm of the mean diameter, or within +2 nm of the mean diameter, or within +1 nm of the mean diameter.

Mean particle sizes disclosed herein, unless clearly indicated otherwise, represent intensity-weighted mean hydrodynamic diameters (also known as the z-average), as determined using dynamic light scattering (DLS). See, for example, ISO 13321.

In some embodiments, metal nanoparticles can comprise coral-shaped metal nanoparticles having a non-uniform cross section and a globular structure formed by multiple, non-linear strands joined together without right angles. In some cases the coral-shaped metal nanoparticles can be used together with spherical-shaped metal nanoparticles (e.g., in order to potentiate the spherical-shaped metal nanoparticles).

In some embodiments, metal nanoparticles can comprises at least one metal selected from the group consisting of gold, platinum, silver, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, and alloys thereof. Nanoparticles comprised of silver, gold, and mixtures and alloys thereof can be particularly effective.

In some embodiments, a method of killing or deactivating a microbe comprises: (1) applying an antimicrobial composition comprising a carrier and metal nanoparticles onto or into a substrate containing microbes, and (2) the antimicrobial composition killing or deactivating the microbes. The substrate can be a living organism or a non-living object.

In some embodiments, a method of deactivating a virus comprises: (1) applying an anti-viral composition comprising metal nanoparticles having a particle size of about 8 nm or less, or about 1 nm to about 7 nm, or about 2 nm to about 6.5 nm, or about 3 nm to about 6 nm onto or into a substrate containing a virus, and (2) the anti-viral composition deactivating the virus.

In some embodiments, a method of deactivating Ebola viruses comprises: (1) applying an anti-viral composition comprising a carrier and metal nanoparticles onto or into an animal or non-living substrate contaminated with Ebola viruses, and (2) the metal nanoparticles deactivating Ebola viruses by attaching to glycoproteins and/or denaturing one or more proteins in the Ebola viruses.

In some embodiments, a method of treating or preventing Ebola virus disease comprises: (1) administering a pharmaceutically acceptable quantity of spherical-shaped nonionic metal nanoparticles having a particle size in a range of about 0.22 nm to about 2 nm to a living organism; and (2) the spherical-shaped nonionic metal nanoparticles deactivating Ebola viruses on or in the living organism.

In some embodiments, a method of treating or preventing Ebola virus disease comprises: (1) administering a pharmaceutically acceptable quantity of spherical-shaped nonionic metal nanoparticles having a particle size in a range of Q: about 2 nm to about 10 nm to a living organism; and (2) the spherical-shaped nonionic metal nanoparticles deactivating Ebola viruses on or in the living organism by binding to, associating with, and/or disrupting the 3CSY glycoproteins of the Ebola viruses.

In some embodiments, a method of killing a bacterium comprises: (1) applying an anti-bacterial composition comprising metal nanoparticles having a particle size of about 8 nm or less, or about 3 nm to about 14 nm, or about 5 nm to about 13 nm, or about 7 nm to about 12 nm, or about 8 nm to about 10 nm onto or into a substrate containing a bacterium, and (2) the anti-bacterial composition killing the bacterium.

In some embodiments, a method of killing a fungus comprises: (1) applying an anti-fungal composition comprising metal nanoparticles having a particle size of about 9 nm to about 20 nm, or about 10 nm to about 18 nm, or about 11 nm to about 16 nm, or about 12 nm to about 15 nm onto or into a substrate containing a fungus, and (2) the anti-fungal composition killing the fungus.

These and other advantages and features of the invention will be set forth in part in the description which follows, and in part will become apparent to those skilled in the art upon examination of the following or may be learned by practice of the invention.

### BRIEF DESCRIPTION OF THE DRAWINGS

FIGS. 1A-1D show TEM images of various non-spherical nanoparticles (i.e., that have surface edges and external bond angles) made according to conventional chemical synthesis or electrical discharge methods;

FIGS. 2A-2C show TEM images of exemplary nonionic spherical-shaped metal nanoparticles (i.e., that have no surface edges or external bond angles), the nanoparticles showing substantially uniform size and narrow particle size distribution, smooth surface morphology, and solid metal cores without the use of coating agents or capping agents;

FIGS. 3A-3C show transmission electron microscope (TEM) images of nonionic “coral-shaped” nanoparticles;

FIGS. 4A-4C schematically illustrated a proposed mechanism of action by which the nanoparticles can kill or deactivate bacteria;

FIG. 5 illustrates the results of conductivity testing comparing various nanoparticle solutions and showing that spherical, metal nanoparticles according to the disclosed embodiments are nonionic; and

FIG. 6 illustrates the results of nanoparticle antimicrobial biofilm testing, demonstrating that the disclosed nonionic nanoparticles were able to effectively penetrate the biofilm and inhibit the bacteria within the film.

### DETAILED DESCRIPTION

Disclosed herein are antimicrobial compositions and methods for killing or deactivating microbes, such as viruses (including Ebola viruses), bacteria, or fungi. In some embodiments, metal nanoparticles are dispersed within or contained on or within a carrier that can be applied onto or into a substrate containing a microbe. The carrier can be a liquid, gel or solid. The antimicrobial compositions can be formulated to selectively and preferentially kill one or more specific microbes. In addition, silver nanoparticles made by laser ablation so as to be nonionic, ground state, and without external edges or bond angles that cause release of metal ions provide unexpectedly superior antimicrobial activity compared to colloidal silver (i.e., silver nanoparticles manufactured by conventional chemical reduction processes, typically with some form of capping agent, which are known to release silver ions).

In some embodiments, antimicrobial compositions may comprise a carrier and a plurality of metal nanoparticles having a particle size and a particle size distribution selected so as to selectively and preferentially kill one of a virus, a bacterium, or a fungus.

In some embodiments, anti-viral compositions comprise metal nanoparticles having a particle size of about 8 nm or less, or about 0.22 nm to about 2 nm, or about 0.22 nm to about 1.5 nm, or about 0.24 nm to about 1 nm, or about 0.27 nm to about 0.7 nm, or about 0.3 nm to about 0.5 nm, or about 0.35 nm to about 0.45 nm, or about 1 nm to about 7 nm, or about 2 nm to about 6.5 nm, or about 3 nm to about 6 nm, or about 2 nm to about 10 nm, or about 4 nm to about 10 nm, or about 6 nm to about 10 nm.

In some embodiments, anti-bacterial compositions can include metal nanoparticles having a particle size of about 3 nm to about 14 nm, or about 5 nm to about 13 nm, or about 7 nm to about 12 nm, or about 8 nm to about 10 nm.

In some embodiments, anti-fungal compositions can include metal nanoparticles having a particle size of about 9 nm to about 20 nm, or about 10 nm to about 18 nm, or about 11 nm to about 16 nm, or about 12 nm to about 15 nm.

Within any of the foregoing size ranges, it is possible to select “designer antimicrobial particles” of specific size that are particularly effective in targeting a specific microbe.

The ability to select and use microbe-specific nanoparticles provides a number of benefits. In the case where only certain nanoparticle sizes are effective in killing a particular microbe or class of microbes, providing metal nanoparticles within a narrow particle size distribution of the correct particle size maximizes the proportion of nanoparticles that are effective in killing the target microbe and minimizes the proportion of nanoparticles that are less effective, or ineffective, in killing the target microbe. This, in turn, greatly reduces the overall amount or concentration of nanoparticles required to provide a desired kill- or deactivation rate of a targeted microbe. Eliminating improperly-sized nanoparticles also reduces the tendency of the composition to kill or harm non-targeted microbes or other cells, such as healthy mammalian or human cells. In this way, highly specific antimicrobial compositions can better target a harmful microbe while being less harmful or even non-toxic to humans, animals, and plants.

### Nanoparticle Configurations

In some embodiments, the metal nanoparticles may comprise or consist essentially of nonionic, ground state metal nanoparticles. Examples include spherical-shaped metal nanoparticles, coral-shaped metal nanoparticles, or a blend of spherical-shaped metal nanoparticles and coral-shaped metal nanoparticles.

In some embodiments, nonionic metal nanoparticles useful for making antimicrobial compositions comprise spherical nanoparticles, preferably spherical-shaped metal nanoparticles having a solid core. The term “spherical-shaped metal nanoparticles” refers to nanoparticles that are made from one or more metals, preferably nonionic, ground state metals, having only internal bond angles and no external edges or bond angles.

This is in contrast to hedron-like, faceted, or crystalline nanoparticles, which are often formed using conventional chemical synthesis methods, even though such nanoparticles are often erroneously described in the art as being “spherical” in shape. The skilled person will thus understand that “spherical” nanoparticles as used herein are formed from a laser ablation process that results in nanoparticles of different morphology and functionality as compared to nanoparticles formed via another process such as chemical synthesis, even if such nanoparticles are erroneously described as “spherical.” Moreover, preferred nanoparticles disclosed herein, including spherical nanoparticles, omit coating agents or capping agents that are frequently required in other nanoparticle systems to halt particle growth and/or prevent agglomeration.

In this way, the spherical nanoparticles are highly resistant to ionization, highly stable, and highly resistance to agglomeration. Such nanoparticles can exhibit a high ζ-potential, which permits the spherical nanoparticles to remain dispersed within a polar solvent without a surfactant, which is a surprising and unexpected result.

In general, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less. Nevertheless, selecting spherical-shaped nanoparticles of specific particle size and narrow particle size distribution can be particularly useful for selectively and preferentially killing or deactivating a particular type of microbe, such as a virus, bacterium, or fungus.

In some embodiments, spherical-shaped nanoparticles can have a particle size distribution such that at least 99% of the nanoparticles have a diameter within 30% of the mean diameter of the nanoparticles, or within 20% of the mean diameter, or within 10% of the mean diameter. In some embodiments, spherical-shaped nanoparticles can have a mean particle size and at least 99% of the nanoparticles have a particle size that is within ±3 nm of the mean diameter, ±2 nm of the mean diameter, or 1 nm of the mean diameter. In some embodiments, spherical-shaped nanoparticles can have a ζ-potential of at least 10 mV, preferably at least about 15 mV, more preferably at least about 20 mV, even more preferably at least about 25 mV, and most preferably at least about 30 mV.

Mean particle sizes disclosed herein, unless clearly indicated otherwise, represent intensity-weighted mean hydrodynamic diameters (also known as the z-average), as determined using dynamic light scattering (DLS). See, for example, ISO 13321. The mean diameter can additionally or alternatively be determined using acoustic spectroscopy methods (e.g., Coulter counter) and/or microscopy techniques (e.g., TEM, SEM).

Examples of methods and systems for manufacturing spherical-shaped nanoparticles are disclosed in U.S. Pat. No. 9,849,512 to William Niedermeyer, incorporated herein by reference. There is, however, no teaching or suggestion in Niedermeyer that the spherical-shaped nanoparticles disclosed therein have antimicrobial properties, much less that such spherical-shaped nanoparticles would possess unexpectedly superior antimicrobial activity compared to colloidal silver nanoparticles made by conventional means. Niedermeyer also fails to disclose or suggest particle sizes and concentrations of spherical-shaped nanoparticles that would be effective against viruses, bacteria, and/or fungi.

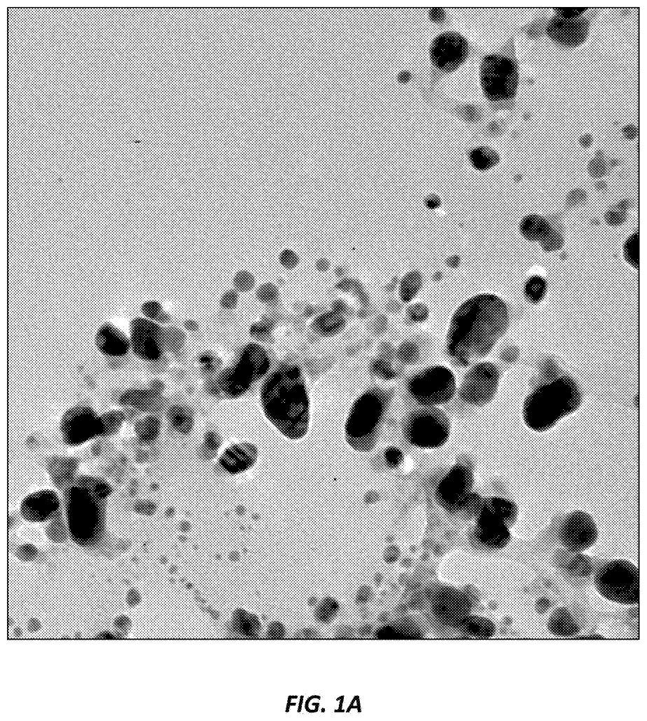

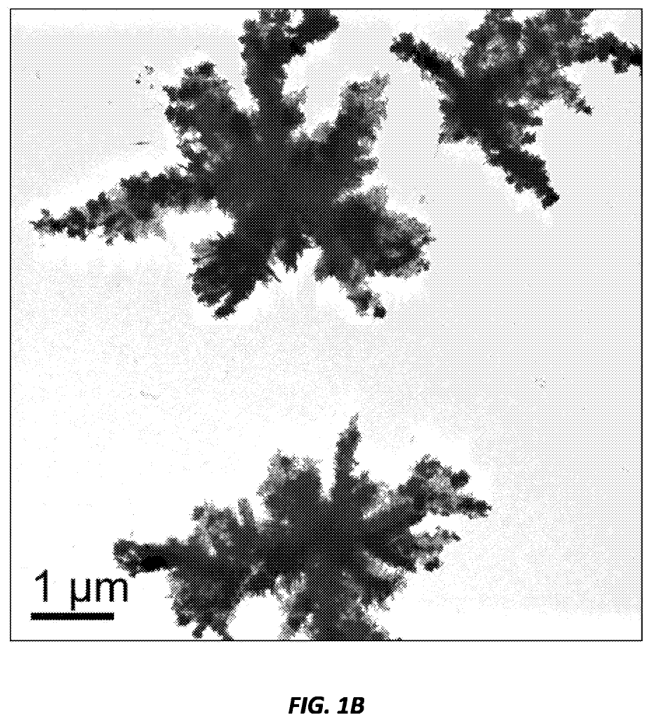

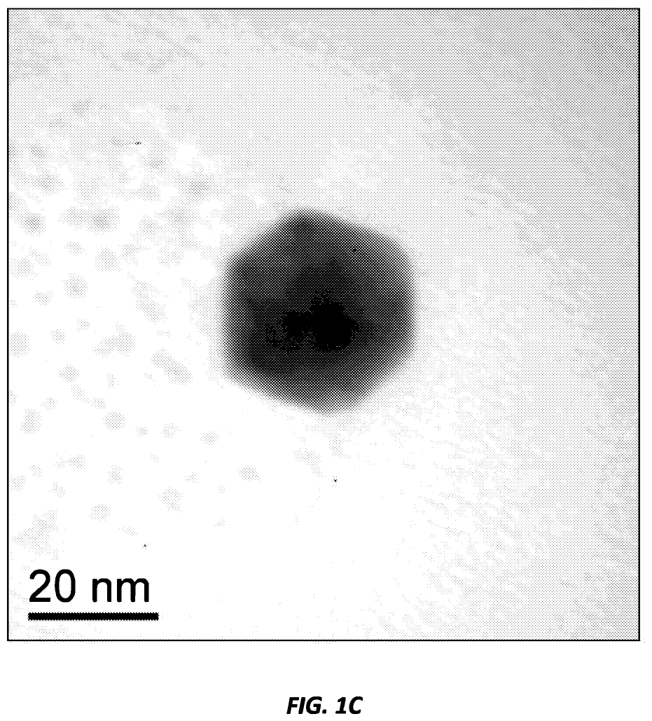

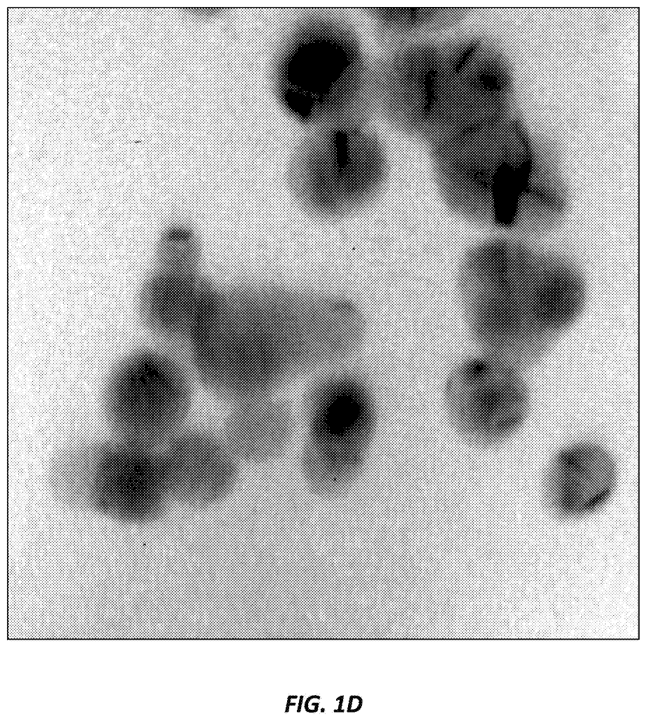

FIGS. 1A-1D show transmission electron microscope (TEM) images of nanoparticles made according to various chemical synthesis methods. As shown, the nanoparticles formed using these various chemical synthesis methods tend to exhibit a clustered, crystalline, faceted, or hedron-like shape rather than a true spherical shape with round and smooth surfaces. They also do not fit the definition of “coral-shaped” nanoparticles,

For example, FIG. 1A shows silver nanoparticles formed using a common trisodium citrate method. The nanoparticles are clustered and have a relatively broad size distribution. FIG. 1B shows another set of silver nanoparticles (available from American Biotech Labs, LLC) formed using another chemical synthesis method and showing rough surface morphologies with many edges. FIG. 1C shows a gold nanoparticle having a hedron shape as opposed to a truly “spherical” shape as that term is used and defined herein. FIG. 1D shows a set of silver nanoparticles (sold under the trade name MesoSilver), which have relatively smoother surface morphologies but are understood to be shells of silver formed over a non-metallic seed material.

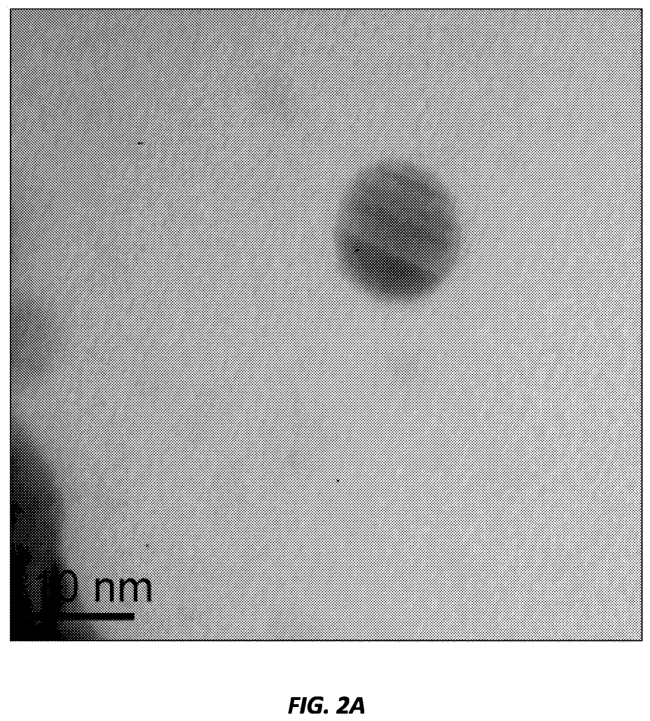

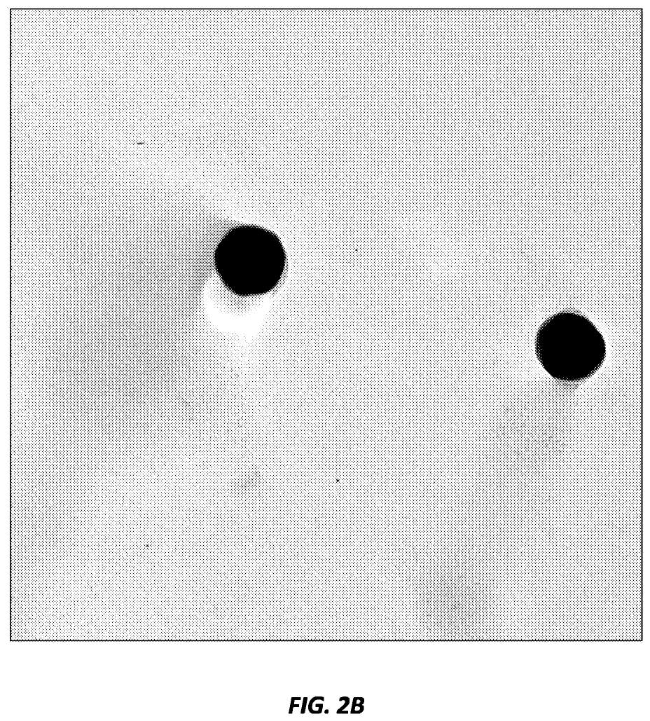

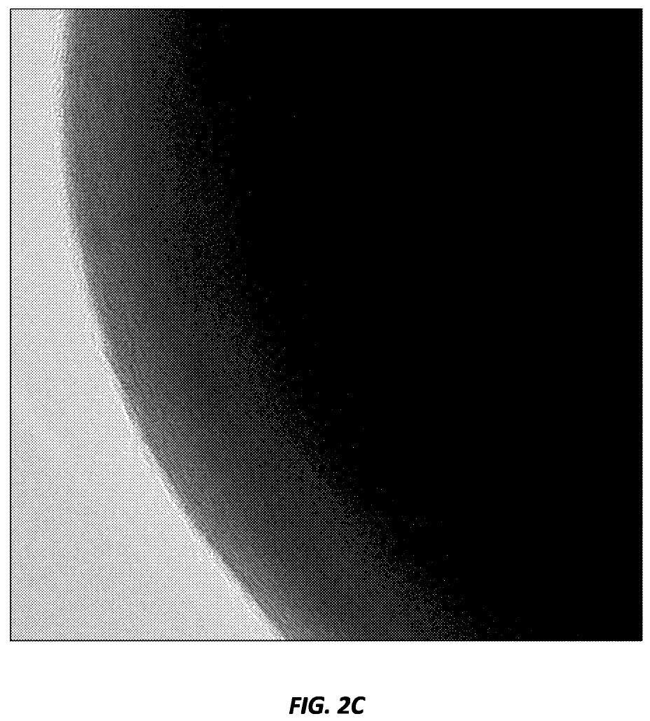

In contrast, the spherical-shaped nanoparticles described herein are solid metal, substantially unclustered, optionally exposed/uncoated (i.e., have no organic capping agent or other organic coating), and have a smooth and round surface morphology along with a narrow size distribution. FIGS. 2A-2C show additional TEM images of spherical-shaped nanoparticles used in the present compositions and methods. FIG. 2A shows a gold/silver alloy nanoparticle (90% silver and 10% gold by molarity). FIG. 2B shows two spherical nanoparticles side by side to visually illustrate size similarity. FIG. 2C shows a surface of a metal nanoparticle showing the smooth and edgeless surface morphology.

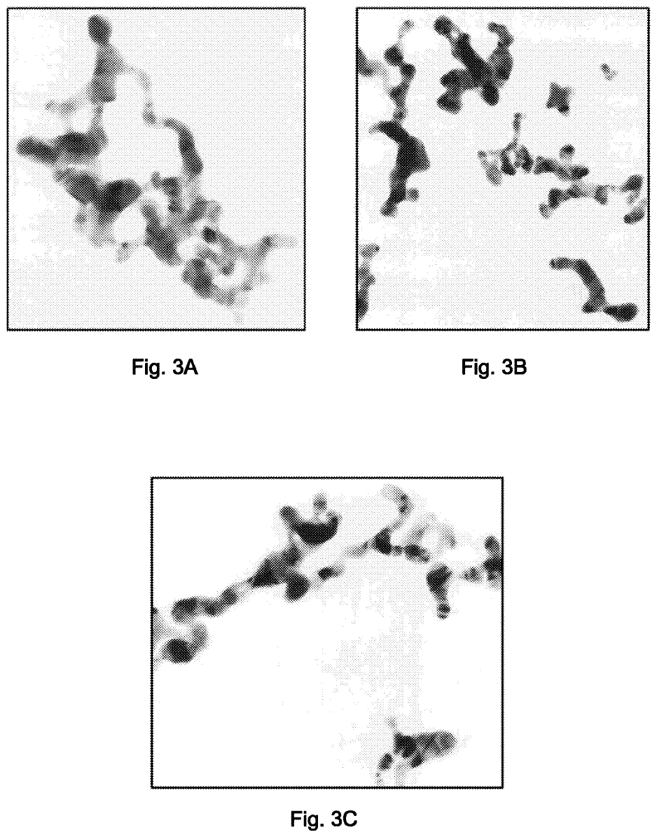

In some embodiments, nonionic metal nanoparticles useful for making nanoparticle compositions may also comprise coral-shaped nanoparticles. The term “coral-shaped metal nanoparticles” refers to nanoparticles that are made from one or more metals, preferably nonionic, ground state metals having a non-uniform cross section and a globular structure formed by multiple, non-linear strands joined together without right angles (see FIGS. 3A-3C). Similar to spherical-shaped nanoparticles, coral-shaped nanoparticles may have only internal bond angles and no external edges or bond angles. Stated differently, such coral-shaped nanoparticles do not have lattice planes or crystalline faces, nor do they depend on crystalline growth based on such structures. This is in contrast to other nanoparticle morphologies such as “nanoflowers” (see, e.g., Sahu et al., “Flower Shaped Nanostructures: An Efficient Bacteria Exterminator” A Search for Antibacterial Agents, Chapter 2, 2007; 73(6): 1712-1720), which include a face centered cubic structure with specified lattice planes. Thus, the term “coral-shaped nanoparticles” excludes the “nanoflowers” disclosed in Sahu et al.

In this way, coral-shaped nanoparticles can be highly resistant to ionization, highly stable, and highly resistance to agglomeration. Such coral-shaped nanoparticles can exhibit a high ζ-potential, which permits the coral-shaped nanoparticles to remain dispersed within a polar solvent without a surfactant, which is a surprising and unexpected result.

In some embodiments, coral-shaped nanoparticles can have lengths ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles can have a particle size distribution such that at least 99% of the nanoparticles have a length within 30% of the mean length, or within 20% of the mean length, or within 10% of the mean length. In some embodiments, coral-shaped nanoparticles can have a ζ-potential of at least 10 mV, preferably at least about 15 mV, more preferably at least about 20 mV, even more preferably at least about 25 mV, and most preferably at least about 30 mV.

Examples of methods and systems for manufacturing coral-shaped nanoparticles are disclosed in U.S. Pat. No. 9,919,363 to William Niedermeyer, which is incorporated by reference. There is, however, no teaching or suggestion in Niedermeyer that the coral-shaped nanoparticles disclosed therein have antimicrobial properties, much less that such coral-shaped nanoparticles would possess unexpectedly superior antimicrobial activity compared to colloidal silver nanoparticles made by conventional means. Niedermeyer also fails to disclose or suggest particle sizes and concentrations of coral-shaped nanoparticles that would provide antimicrobial activity against any of viruses, bacteria, or fungi.

The metal nanoparticles, including spherical-shaped and coral-shaped nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, or alloys thereof.

According to some embodiments, the antimicrobial metal nanoparticles will comprise at least one of silver or gold. In the case of larger metal nanoparticles for use in killing bacteria and fungi, metal nanoparticles may primarily or exclusively comprise silver. However, in the case of smaller metal nanoparticles for use in killing viruses, including Ebola viruses, metal nanoparticles may primarily or exclusively comprise gold. Due to the nature silver and gold atoms making up the nanoparticles, it has been found that gold nanoparticles are better able to hold together at very small sizes (e.g., smaller than about 5-7 nm) compared to silver nanoparticles. On the other hand, in some embodiments, a gold-silver alloy provides the particle stabilizing activity of gold and potentially higher anti-viral activity of silver.

### Antimicrobial Activity

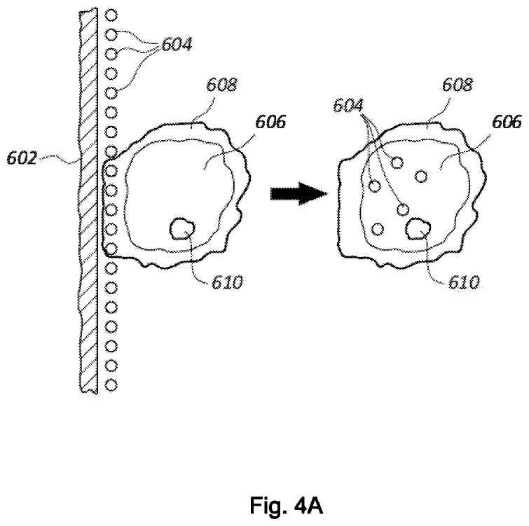

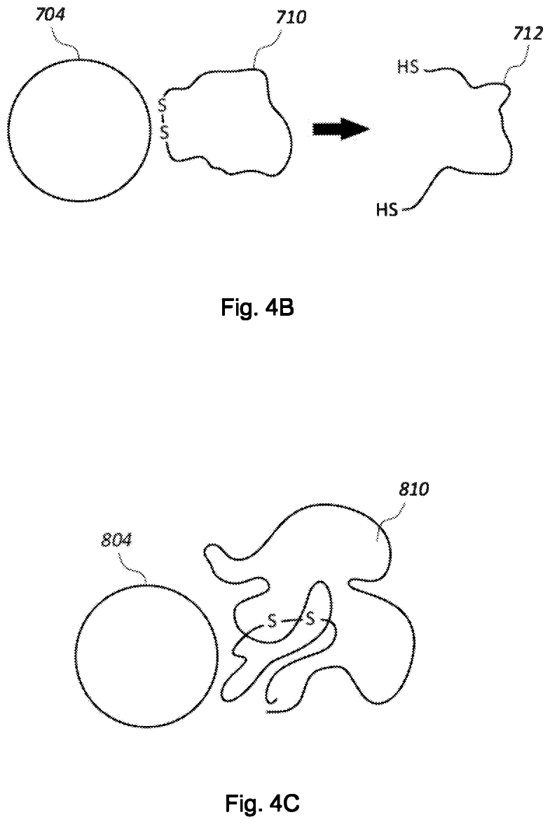

FIG. 4A schematically illustrates a bacterium 608 having absorbed spherical-shaped nanoparticles 604 from a substrate 602 (e.g., from a mucus layer), such as by active absorption or other transport mechanism. The nanoparticles 604 can freely move throughout the interior 606 of bacterium 608 and come into contact with one or more vital proteins or enzymes 610 that, if denatured, will kill or disable the bacterium. A similar mechanism may function where viral or fungal pathogens are involved. Unlike most conventional antibiotics, the nanoparticles effectively kill or deactivate the bacterium without significantly disrupting the cell wall and therefore without significant lysing of the bacteria coming into contact with the nanoparticles.

In addition, silver nanoparticles made by laser ablation so as to be nonionic, ground state, and without external edges or bond angles that cause release of metal ions provide unexpectedly superior antimicrobial activity compared to colloidal silver (i.e., silver nanoparticles manufactured by conventional chemical reduction processes, typically with some form of capping agent, which are known to release silver ions). Such silver nanoparticles made by laser ablation are able to kill bacteria and other microbes without the release of silver ions. This is a surprising, unexpected, and unpredictable result since the prevailing understanding by those skilled in the art is that silver nanoparticles provide antimicrobial activity through the release of silver ions.

Mohamed, et al. “Antimicrobial Activity of Silver-Treated Bacteria against Other Multi-Drug Resistant Pathogens in Their Environment,” Antibiotics (Basel), 2020 Apr. 15; 9(4):181; doi: 10.3390/antibiotics9040181, states: “Silver nanoparticles have an advantage over other antimicrobial nanoparticles in terms of more effective antimicrobial activity, which is achieved as silver ions are released from the crystalline core which triggers chemical disequilibrium to occur within bacterial cells.” Dominguez, et al., “Antibacterial Activity of Colloidal Silver against Gram-Negative and Gram-Positive Bacteria,” Antibiotics (Basel), 2020 Jan. 19; 9(1):36; doi: 10.3390/antibiotics9010036, states: “One of the main characteristics of silver is its oligodynamic effect, which is defined as the high microbicidal capacity of silver ions in water at a very low concentration (one part per million). Silver is an inert metal in its metallic form; however, it is biologically active when it is in the ionic monoatomic state (Ag+) soluble in an aqueous environment (water or tissue fluids). This activated ion shows a strong affinity for sulfhydryl groups and protein residues present in cell membranes.” “How silver ions kill bacteria,” ScienceDaily, https://www.sciencedaily.com/releases/2020/04/200409140021.htm, states: “The researchers observed that silver ions [released by elemental silver) were causing paired strands of DNA in the bacteria to separate, and the binding between the protein and the DNA to weaken.”

For example, one way that the disclosed nanoparticles made by laser ablation may kill or denature a microbe is by catalyzing the cleavage of disulfide (S—S) bonds within a vital protein or enzyme. FIG. 4B schematically illustrates a microbe protein or enzyme 710 with disulfide bonds being catalytically denatured by an adjacent spherical-shaped nanoparticle 704 to yield denatured protein or enzyme 712. In the case of bacteria or fungi, the cleavage of disulfide bonds and/or cleavage of other chemical bonds of vital proteins or enzymes may occur within the cell interior and thereby function to kill the microbe in this manner without causing significant lysis. Such catalytic cleavage of disulfide (S—S) bonds is facilitated by the generally simple protein structures of microbes, in which many vital disulfide bonds are on exposed and readily cleaved by catalysis.

Another potential mechanism by which metal (e.g., silver) nanoparticles made by laser ablation can kill microbes is through the production of active oxygen species, such as peroxides, which can oxidatively cleave protein bonds, including but not limited to amide bonds.

In the case of viruses, spherical-shaped and coral-shaped metal nanoparticles made by laser ablation can alternatively deactivate viruses by attaching to glycoproteins and/or catalyzing protein denaturing reactions in the protein coat so that the virus is no longer able to attach to a host cell and/or inject genetic material into the host cell. Because very small nanoparticles can pass through a virus, denaturing of the protein coat may occur within the interior of the virus. A virus that is rendered unable to attach to a host cell and/or inject genetic material into the host cell is essentially inactive and no longer pathogenic.

Notwithstanding the lethal nature of nonionic metal nanoparticles made by laser ablation relative to microbes, they can be relatively harmless to humans, mammals, and healthy mammalian cells, which contain much more complex protein structures compared to simple microbes in which most or all vital disulfide bonds are shielded by other, more stable regions of the protein. FIG. 4C schematically illustrates a mammalian protein 810 with disulfide (S—S) bonds that are shielded so as to resist being catalytically denatured by an adjacent spherical-shaped nanoparticle 804. In many cases the nonionic nanoparticles do not interact with or attach to human or mammalian cells and can be quickly and safely expelled through the urine without damaging kidneys or other cells, tissues, or organs.

The metal nanoparticles made by laser ablation kill bacteria without significant release of silver (Ag+) or other metal ions. Because the metal nanoparticles do not release significant quantities of silver or other metal ions, they are essentially non-toxic to humans and other animals (i.e., whatever amount or concentration of ions, if any, that are released from the metal nanoparticles is/are below a threshold toxicity level at which they become toxic to humans, other mammals, birds, reptiles, fish, and amphibians).

In the particular case of silver (Ag) nanoparticles made by laser ablation, the interaction of the silver (Ag) nanoparticle(s) within a microbe has been demonstrated to be particularly lethal without the need to rely on the production of silver ions (Ag⁺) to provide the desired antimicrobial effects, as is typically the case with conventional colloidal silver compositions and typical silver nanoparticle compositions. The ability of silver (Ag) nanoparticles to provide effective microbial control without any significant or actual release of toxic silver ions (Ag⁺) into the patient or the surrounding environment is a substantial advancement in the art. Whatever amount or concentration of silver ions released by silver nanoparticles, if any, is well below known or inherent toxicity levels for animals, such as mammals, birds, reptiles, fish, and amphibians.

As used with respect to ion release, the modifying term “significant” means that the effect the term is modifying is clinically noticeable and relevant. Thus, the phrase “without significant release of silver ions” means that though there may technically be some small amount of detectable ion release, the amount is so small as to be clinically and functionally negligible. Similarly, the phrase “without significant cell lysis” means that although there may be some observable cell lysis, the amount is negligible and only tangentially related to the actual primary mechanism of cell death/deactivation.

In contrast, other nanoparticle systems primarily rely on the release of ions for antimicrobial effects. Even though other mechanisms of action are proposed, antimicrobial action via ion release is ““one of the most widely accepted” mechanisms of action.” (See Rhim et al. “Preparation and characterization of bio-nanocomposite films of agar and silver nanoparticles: laser ablation method,” Carbohydrate Polymers 103 (2014) 456-465). Thus, even if other mechanisms of action may be involved in the antimicrobial activity of silver nanoparticles used in prior methods, the skilled person would not expect particles that do not release silver ions to provide antimicrobial activity to the same degree (or greater degree) than nanoparticles that presumably provide such other mechanisms of action in addition to ion release. In other words, if multiple mechanisms of action are assumed, removing “one of the most widely accepted” mechanisms of action would be expected to lower overall efficacy, even if the other mechanisms remained. One of skill in the art would therefore expect silver nanoparticles that release silver ions to have greater antimicrobial efficacy than silver nanoparticles that are nonionic and do not release any significant amount of silver ions. Surprisingly, however, as shown in the examples herein, nonionic silver nanoparticles made via laser ablation were shown to provide greater antimicrobial efficacy as compared to a standard silver nanoparticle formulation, and even to show antimicrobial efficacy on par with silver nitrate (which is fully ionic).

As discussed herein, the size of the nanoparticles can be selected to target and selectively kill specific types of microbes, including Ebola viruses. By way of further example, sub-micron sized metal nanoparticles (e.g., gold or gold-silver alloy nanoparticles having a diameter of about 0.4 nm) may be most effective in killing Ebola viruses, which have an oddly elongated and looped configuration that can potentially shield vital proteins from larger nanoparticles. Using very small nanoparticles as described herein, particularly nonionic metal nanoparticles, permits the nanoparticles to more easily penetrate into and promote denaturing reactions within the protein coat, such as cleavage of disulfide (S—S) bonds in capsid proteins of the virus. Additionally, or alternatively, such nanoparticles can also bind to glycoproteins of the protein coat. For example, nanoparticles having a size of about 2 nm to about 10 nm, or about 4 nm to about 10 nm, or about 6 nm to about 10 nm can bind and/or cleave the 3CSY glycoprotein located on the surface of the Ebola virus. Either or both mechanisms can prevent a virus from attaching to a host cell and introducing its genetic material therein. An Ebola virus that is rendered unable to attach to a host cell and/or inject genetic material into the host cell is essentially inactive and no longer pathogenic.

The inventors have also discovered that nonionic metal nanoparticles used in the disclosed compositions, including spherical-shaped silver nanoparticles formed by laser ablation, are less likely to result in antimicrobial resistance. This contrasts with colloidal silver and other silver nanoparticles made by chemical processes, which have external bond angles and facets and typically provide antimicrobial activity by releasing silver ions. Some studies have shown microbial resistance to ionic silver in as few as 6 generations, whereas spherical-shaped silver nanoparticles formed by laser ablation and incorporated into the disclosed antimicrobial compositions and used in the disclosed methods have been shown to have stable antimicrobial activity even after 28 passages, with no diminution of antimicrobial activity, including no significant reduction in the MIC (minimum inhibitory concentration) for a given microbe. See Example 14 disclosed herein. See also Sharif, et al, Escaping the ESKAPE Antimicrobial Resistant Cycle with EVQ-218, (https://doi.org/10.3390/1010000), which is incorporated herein by reference.

When antimicrobial resistance to colloidal silver and other silver ion-releasing silver nanoparticles occurs, increasing concentrations of such nanoparticles are necessary to maintain the ability to kill microbes. However, silver ions, particularly at higher concentrations, can also be toxic to humans, mammals, fish, birds, and other higher-level organisms

### Microbe-Specific Nanoparticles

### Anti-Viral Nanoparticles

In some embodiments, spherical-shaped nanoparticles designed to selectively and preferentially deactivate viruses can have a diameter of about 8 nm or less, such as about 1 nm to about 7 nm, or about 2 nm to about 6.5 nm, or about 3 nm to about 6 nm, or a mean diameter within a range defined using any two of the foregoing values as endpoints. Within these size ranges it is possible to select “designer anti-viral particles” of specific size that are particularly effective in targeting a specific virus. Spherical-shaped nanoparticles within the foregoing ranges, particularly which are smaller than about 6 nm, are significantly less effective in killing fungi and bacteria than the differently sized particles discussed herein that target such other microbes.

### Anti-Bacterial Nanoparticles

In some embodiments, spherical-shaped nanoparticles designed to selectively and preferentially deactivate bacteria can have a mean diameter of about 3 nm to about 14 nm, or about 5 nm to about 13 nm, or about 7 nm to about 12 nm, or about 8 nm to about 10 nm, or a mean diameter within a range defined using any two of the foregoing values as endpoints. Within these size ranges it is possible to select “designer anti-bacterial particles” of specific size that are particularly effective in targeting a specific bacterium. Spherical-shaped nanoparticles within the foregoing ranges, particularly which are in a range of about 8 nm to about 10 nm, are significantly less effective in killing viruses and fungi than the differently sized particles discussed herein that target such other microbes.

Spherical-shaped nanoparticles formed by laser ablation have proven to be effective in killing or deactivating both gram negative and gram positive bacteria, including Candidatus Liberibacter spp., Clostridium spp., Clostridium botulinum, Acinetobacter spp., Acinetobacter baumannii, Enterobacter spp., Enterococcus faecium, Staphylococcus spp., Staphylococcus aureus, Klebsiella spp., Klebsiella pneumoniae, Acinetobacter baumannii, Pseudomonas spp., Pseudomonas aeruginosa, Escherichia spp., Escherichia Coli, Enterobacter cloacae, S. typhimurium, Bacillus subtilis, K. pneumoniae, Serratia marcescens, Salmonella spp., Shigella spp, Proteus spp., Serratia spp., Burkholderia spp., and Stenotrophomonas spp.

### Anti-Fungal Nanoparticles

In some embodiments, spherical-shaped nanoparticles designed to selectively and preferentially deactivate fungi can have a mean diameter of about 9 nm to about 20 nm, or about 10 nm to about 18 nm, or about 11 nm to about 16 nm, or about 12 nm to about 15 nm, or about 13 nm to about 14 nm, or a mean diameter within a range defined using any two of the foregoing values as endpoints (e.g., 13 nm to 20 nm). Within these size ranges it is possible to select “designer anti-fungal particles” of specific size that are particularly effective in targeting a specific fungus. Spherical-shaped nanoparticles within the foregoing ranges, particularly which are larger than about 12 nm, are significantly less effective in killing viruses and bacteria than the differently sized particles discussed herein that target such other microbes.

According to some embodiments, the antimicrobial metal nanoparticles will comprise at least one of silver or gold. In the case of larger metal nanoparticles for use in killing bacteria and fungi, the metal nanoparticles may primarily or exclusively comprise silver. However, in the case of smaller metal nanoparticles for use in killing viruses, the metal nanoparticles may primarily or exclusively comprise gold. Due to the nature of silver and gold atoms making up the nanoparticles, it has been found that gold nanoparticles are better able to hold together at very small sizes (e.g., smaller than about 5-7 nm) compared to silver nanoparticles. On the other hand, a gold-silver alloy provides the particle stabilizing activity of gold and the higher microbe killing activity of silver.

### Multi-Component Nanoparticle Compositions

In some embodiments, coral-shaped metal nanoparticles can be used in conjunction with spherical-shaped metal nanoparticles. In general, spherical-shaped metal nanoparticles can be smaller than coral-shaped metal nanoparticles and in this way can provide very high surface area for catalyzing desired reactions or providing other desired benefits. On the other hand, the generally larger coral-shaped nanoparticles can exhibit higher surface area per unit mass compared to spherical-shaped nanoparticles because coral-shaped nanoparticles have internal spaces and surfaces rather than a solid core and only an external surface. In some cases, providing antimicrobial compositions containing both spherical-shaped and coral-shaped nanoparticles can provide synergistic results. For example, coral-shaped nanoparticles can help carry and/or potentiate the activity of spherical-shaped nanoparticles in addition to providing their own unique benefits.

In some embodiments, the antimicrobial compositions may include both spherical-shaped and coral-shaped nanoparticles. In some embodiments, the mass ratio of spherical-shaped nanoparticles to coral-shaped nanoparticles in the nanoparticle composition can be in a range of about 1:1 to about 50:1, or about 2.5:1 to about 25:1, or about 5:1 to about 20:1, or about 7.5:1 to about 15:1, or about 9:1 to about 11:1, or about 10:1. The particle number ratio of spherical-shaped nanoparticles to coral-shaped nanoparticles in the nanoparticle composition can be in a range of about 10:1 to about 500:1, or about 25:1 to about 250:1, or about 50:1 to about 200:1, or about 75:1 to about 150:1, or about 90:1 to about 110:1, or about 100:1.

### Carriers

In some embodiments, a nanoparticle composition includes a carrier for C delivering the metal nanoparticles onto or into a living or non-living substrate. The carrier can be a liquid, gel, or solid. Some carriers may be more suitable than others depending on the living or non-living substrate being treated. For example, the solubility characteristics of the carrier can be selected to maximize or otherwise provide a desired diffusion throughout a treated organism part and/or another portion of the organism in contact with the treated organism part.

Examples of compounds that can be utilized for topical applications and can be used as carriers include, but are not limited to, water, alcohols, ketones, esters, citrus oils, essential oils, vegetable and other plant and natural oils, triglycerides, ethers, organic solvents, methanol, ethanol, isopropyl alcohol, other alcohols, glycols, glycerin, polyols, 1,3-propandiol, DMSO, petroleum jelly, waxes, polymers, polymerizable materials, and surfactants.

In one embodiment, the carrier is a cream or lotion including a glycerin and/or stearic acid cream base optionally containing oils such as coconut oil, olive oil, grape seed oil, shea butter, mango butter, and/or vitamin E oil along with an emulsifying wax.

In other embodiments the carrier is a water or combined water and alcohol solution which itself contains a micro to millimolar concentration of a separate stabilizing agent dissolved into the carrier so as to maintain the nanoparticles within the overall composition.

Exemplary carriers for nasal or pulmonary aerosol or inhalation administration include solutions in saline which can contain, for example, benzyl alcohol or other suitable preservatives, absorption promoters to enhance bioavailability, and/or other solubilizing or wetting or dispersing agents, such as glycerin, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g. dextran sulfate); and glycoaminoglycans and glycosaminoglycan-like compounds (e.g., hyaluronic acid), for example. In some embodiments, the nanoparticles and additional stabilizing agents and/or carriers are formulated as dry powders (e.g., powders useful for administering with dry powder inhalers).

Exemplary aerosols useful for nasal and/or inhalation administration include a vaporizable propellant, such as low molecular weight hydrofluorocarbons or hydrocarbons that are liquid when constrained in a suitable container and are biocompatible and non-irritating. Ingredients such as water, alcohol, propylene glycol, and polyethylene glycols can be additionally included. Other embodiments, also useful for nasal and/or inhalation administration, are provided as sprays (e.g., omitting an aerosol propellant). Such spray formulation may be provided as a solution, suspension, or emulsion capable of forming a fine mist for administration, and in some embodiments, may include saline and/or be isotonic.

Exemplary injectable solutions include an aqueous emulsion or oleaginous suspension or saline solution (e.g., isotonic, hypotonic, or hypertonic, optionally including dextrose and/or other electrolytes or additives). Such compositions can also include suitable dispersing or wetting agents. The sterile injectable preparation may also be formed in a non-toxic parenterally acceptable diluent or solvent, such as a solution in 1,2-propanediol (propylene glycol). Additional examples include solutions or suspensions which can contain, for example, suitable non-toxic diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Gels known in the art can be used as carriers, such as gels containing one or more of the foregoing liquid components together with known gelling agents. Gel compositions can more easily adhere to a living or non-living substrate being treated. An exemplary gel carrier can include mineral oil gelled with polyethylene.

Solid carriers can be used for different reasons, such as to elute nanoparticles into an organism over time. Examples of solid carriers include, but are not limited to, polymers, rubbers, elastomers, foams, gums, and polymer coatings applied to medical devices or other substrates. Depending on the characteristics of the organism to be treated and the desired rate of elution, one of skill in the art can select an appropriate solid carrier material.

In some embodiments, an antimicrobial composition can be formulated so that the metal nanoparticles are included in a concentration so that a measured quantity of the nanoparticle composition, when applied onto or into an organism or organism part, will provide a predetermined concentration or quantity of metal nanoparticles. The nanoparticle composition can have a higher concentration of nanoparticles that become diluted when mixed with other liquids applied to or naturally contained within the organism or organism part. Depending on the organism or organism part being treated, the nature of the nanoparticles being added, and the type of carrier being used, the antimicrobial composition may contain about 10 ppb (parts per billion) to about 100 ppm (parts per million) by weight of the antimicrobial composition, or about 15 ppb to about 90 ppm, or about 100 ppb to about 75 ppm, or about 500 ppb to about 60 ppm of metal nanoparticles by weight, or about 1 ppm to about 50 ppm, or about 2 ppm to about 25 ppm, or about 3 ppm to about 20 ppm metal nanoparticles by weight of the antimicrobial composition.

In some embodiments, the antimicrobial composition can also include one or more optional components or adjuvents to provide desired properties, including, but not limited to food, vitamins, minerals, antimicrobial agents, electrolytes, moisturizers, emollients, antiseptics, and/or plant extracts.

In some embodiments, the carrier may also function as, or may include, a stabilizing agent. For example, in some embodiments it may be desirable to have different specifically sized nanoparticles within the same solution to take advantage of each of the different properties and effects of the different particles. However, when differently sized particles are mixed into a single solution, the overall long-term stability of these particles within that single solution may be substantially diminished as a result of unequal forces exerted on the various particles causing eventual agglomeration of the particles. This phenomenon may become even more pronounced when that solution is either heated or cooled significantly above or below standard room temperature conditions.

Examples of stabilizing agents include alcohols (e.g., ethanol, propanol, butanol, etc.), polyphenols (e.g., arjuna bark extract, grape seed extract, etc.), mono-glycerides, di-glycerides, or triglycerides (e.g., grape seed oil, coconut oil, and the like), oils (e.g., lavender), other terpenes, amine compounds (e.g., mono-, di-, or tri-ethanol amine), carbohydrates (e.g., sucrose, fructose), liposomes, creams, other emulsions, and other polymers.

In some embodiments, stabilizing agents are dissolved within a separate carrier in the micro- to milli-molar concentration range with the upper range limitation typically being constrained not by efficacy but by product cost.

These various stabilizing agents have the capacity to hold the at least two differently sized and/or shaped nanoparticles in suspension and deliver these nanoparticles into the treatment area of a person or animal without so powerfully retaining the nanoparticles so as to diminish the antimicrobial properties of the nanoparticles.

### Treatment Methods

In some embodiments, a method of killing or deactivating a microbe comprises: (1) applying an antimicrobial composition comprising a carrier and metal nanoparticles onto or into a substrate containing microbes; and (2) the antimicrobial composition killing or denaturing the microbes. The substrate can be a living organism, such as an organism that is infected with, or may become infected with, a disease caused by a microbe. Alternatively, the substrate can be a non-living object that has come into contact, or is at risk of coming into contact, with a disease causing or otherwise unwanted microbe, such as clothing, bedding, wound dressings, medical devices or implants, food, food containers, intravenously injectable liquids, farm equipment, animal feeding devices, watering troughs, and the like.

In some embodiments, metal nanoparticles kill bacteria or fungi by entering the cell and catalyzing protein denaturing reactions. In some embodiments, metal nanoparticles “deactivate” viruses by attaching to glycoproteins and/or catalyzing protein denaturing reactions in the protein coat. Examples of protein denaturing reactions include reactions involving one or more of disulfide (S—S) bond cleavage and formation of active oxygen species that attack amide or other bonds.

In some embodiments, a method of deactivating a virus comprises: (1) applying an anti-viral composition comprising metal nanoparticles having a selected particle size (e.g., having a mean diameter in a range of about 1 nm to about 7 nm) onto or into a substrate containing a virus, and (2) the anti-viral composition deactivating the virus.

In some embodiments, a method of deactivating Ebola viruses comprises: (1) applying an anti-viral composition comprising a carrier and metal nanoparticles onto or into an animal or non-living substrate contaminated with Ebola viruses; and (2) the metal nanoparticles deactivating Ebola viruses by attaching to glycoproteins and/or denaturing one or more proteins in the Ebola viruses. The substrate can be a living organism, such as an organism that is infected with, or may become infected with, Ebola viruses. Alternatively, the substrate can be a non-living object that can come into contact with an organism and potentially cause or spread disease, such as clothing, beading, wound dressing, medical device or implant, food, IV liquid, farm equipment, animal feeding devices, watering troughs, and the like.

In some embodiments, metal nanoparticles deactivate viruses by attaching to glycoproteins and/or catalyzing protein denaturing reactions in the protein coat. Examples of protein denaturing reactions include reactions involving one or more of disulfide (S—S) bond cleavage and formation of active oxygen species that attack amide or other bonds.

In some embodiments, the method may comprise administering a pharmaceutically acceptable quantity of the anti-viral composition to a mammal in order to treat or prevent Ebola virus disease. The anti-viral composition can be administered in any appropriate fashion, including but not limited to, intravenously, via inhalation, orally, and/or topically. Advantageously, the metal nanoparticles selectively deactivate Ebola viruses without harming humans or animals contacting the metal nanoparticles.

In some embodiments, the method may comprise applying the anti-viral composition to one or more of medical equipment, clothing, bandages, waste receptacles, syringes, syringe needles, inhalation equipment, or implants.

In some embodiments, a method of treating or preventing Ebola virus disease comprises: (1) administering a pharmaceutically acceptable quantity of spherical-shaped nonionic metal nanoparticles having a particle size in a range of about 0.22 nm to about 2 nm to a living organism, and (2) the anti-viral composition deactivating the virus.

In some embodiments, a method of killing a bacterium comprises: (1) applying an anti-bacterial composition comprising metal nanoparticles having a selected particle size (e.g., having a mean diameter in a range of about 8 nm to about 10 nm) onto or into a substrate containing a bacterium, and (2) the anti-bacterial composition killing the bacterium.

In some embodiments, a method of killing a fungus comprises: (1) applying an anti-fungal composition comprising metal nanoparticles having a selected particle size (e.g., having a mean diameter in a range of about 12 nm to about 15 nm) onto or into a substrate containing a fungus, and (2) the anti-bacterial composition killing the fungus.

### EXAMPLES

In the following examples, the nonionic, ground state, uncoated metal nanoparticles described above may be referred to as “Attostat” or (Evoq Nano) nanoparticles, “Niedermeyer” nanoparticles, “Attostat Ag,” “Evoq Nano” particles, or the like. Except where noted otherwise, the Attostat/Evoq Nano nanoparticles utilized were spherical, silver nanoparticles having a mean diameter ranging from 6 nm to 10 nm.

### Example 1

Testing measured the transepithelial electrical resistance (TER) of a silver nanoparticle composition applied to the apical surface of cystic fibrosis patient derived primary cultures of bronchial epithelia (maintained in ALI cultures). TER is a measure of epithelial tight junction integrity which underlies the physical barrier function of airway epithelia. Changes in TER of 12 epithelia were observed over 24 hours.

Changes in measured TER of epithelia treated with spherical, nonionic, ground state silver nanoparticle formulations at 3 ppm were not significantly different from the responses to vehicle treatment at the sampled time points, as determined with ANOVA and Turkey-Kramer HSD post-test analysis with P<0.05. The impact of the silver nanoparticle formulations on the barrier function of well differentiated primary CF bronchial epithelia was therefore not distinct from the impact of vehicle treatment.

Further, there were no visually distinct differences in microscopic appearance of the epithelia at about 100× magnification in a phase contrast microscope. Ciliary activity was also similar across treatment groups. These results were surprising given the general belief that silver nanoparticles of such size would release silver ions and be toxic to such cells.

### Example 2

This test compared the effect on zebra fish of nonionic, ground state silver nanoparticles formed via laser ablation compared to other silver nanoparticles (i.e., colloidal silver) formed through conventional chemical synthesis or electrolysis methods, silver nitrate, and a control tank with plain water. The colloidal silver nanoparticles formed through a chemical synthesis process and the nanoparticles formed through an electrolysis process both caused the fish to exhibit signs of toxicity, including death, slowed movement and settling near the bottom of the tank. This was likely due to the release of silver ions from the colloidal silver nanoparticles. The nanoparticles formed through an electrolysis process and the silver nitrate both killed the fish within 2 hours of exposure, also likely due to the release of silver ions.

In contrast, the zebra fish in the tank treated with the nonionic, ground state silver nanoparticles of the present application made by laser ablation and the fish in the control tank were equally healthy and active. None of the zebrafish exposed to the nonionic, ground state silver nanoparticles of the present application made by laser ablation died during the course of the study, whereas all other treatments were associated with at least some zebrafish death.

The results of the zebra fish study were surprising in light of the general knowledge that silver nanoparticles show toxicity in such studies. For example, the authors of Mansouri et al., “Effects of Short-Term Exposure to Sublethal Concentrations of Silver Nanoparticles on Histopathology and Electron Microscope Ultrastructure of Zebrafish (Danio rerio) Gills,” Iranian J. Toxicity, Vol. 10, No 1, January-February 2016, state the concern that “[t]he increasing use of nanomaterials and nanoproducts has increased the possibility of contamination of the environment, which may have adverse effects on different organisms” (Abstract). The authors concluded, following the study, that “[b]ased on the adverse effects of AgNPs [colloidal silver nanoparticles] on zebrafish gills, silver nanoparticle solutions can be hazardous pollutants for the environment” (page 15).

### Example 3

Neutrophil testing was done using complete blood count (CBC) analysis. Testing 0.2 and 1.0 μg/mL (i.e., 0.2 ppm and 1.0 ppm)) of Attostat Ag nanoparticles showed that after up to 6 hours exposure time, no blood panel values deviated from normal ranges. At 24 hours exposure time, both control and test samples showed borderline values for MCHC (Mean Corpuscular Hemoglobin Concentration, just below minimum normal range) and MPV (Mean Platelet Volume, just above the maximum normal range).

The only deviation from normal values occurred in the 0.2 μg/mL Attostat Ag nanoparticle sample, which exhibited very slight elevation in EOS % (Eosinophil Percentage, just above maximum normal range). Overall, these results show no significant toxicological effects on the full spectrum of blood cells and components. This is particularly encouraging as forecasted therapeutics typically would not exceed 8-10 μg/mL (i.e., 8-10 ppm), resulting in much lower local concentrations throughout the bloodstream and other portions of the body.

### Example 4

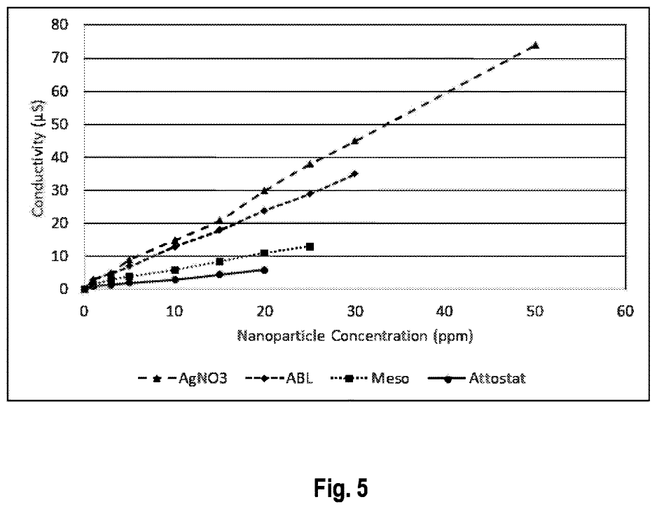

FIG. 5 illustrates the results of conductivity testing comparing various nanoparticle solutions. In the Figure, “Attostat” corresponds to spherical-shaped, nonionic silver nanoparticles formed by laser ablation such as described herein, “AgNO₃” is silver nitrate, “Meso” represents a commercially available silver nanoparticle formulation with nanoparticles formed through a chemical reduction process, and “ABL” represents a commercially available silver nanoparticle formulation understood to be formed through an electrolysis process.

The results illustrate that the Attostat silver nanoparticle formulation had significantly less ion release than any of the other tested nanoparticle formulations. It should be noted that the measured conductivity for Attostat silver nanoparticle formulations, even at the highest measured concentration of 16 ppm, remained low enough to be on par with typical conductivity measurements for high quality deionized water.

### Example 5

An antibacterial efficacy test was carried out comparing a “Niedermeyer” silver nanoparticle formulation (8 nm size) against silver nitrate and against the National Institute of Standards and Technology (NIST) Standard Nanocomposix 10 nm silver nanoparticles. The NIST nanoparticles are formed by a chemical reduction process that utilizes citrate as reducing and capping agent. The NIST nanoparticles have a conductivity similar to the “Meso” nanoparticles of Example 4, with detectable but low levels of silver ions.

Relative Light Unit (RLU) counts were recorded at 12 hours and 24 hours post treatment. RLU measurements were carried out using a Hygiena SystemSURE Plus V.2 SN067503 RLU meter with Hygenia AquaSnap TOTAL ATP Water Test Cat #U143 Lot #153019. Culturing media was Hardy Diagnostics Buffered Peptone Water Lot #118272. Samples were prepared with the nanoparticle treatments and then diluted with the media to provide the tested concentrations. The test organism (Microbiologics, E. coli, KwikStik, ATCC #51813, Ref #0791 K, Lot #791-1-6) was incubated in fresh Buffered Peptone Water growth media for 24 hours prior to exposure to the nanoparticle treatments. Tables 1 and 2 illustrate results of RLU counts 12 and 24 hours post nanoparticle treatment, respectively.

TABLE 1
RLU Counts at 12 Hours Post Exposure to Nanoparticle Treatment

| | Attostat 8 nm | NIST Standard | AgNO3 Silver |
|---|---|---|---|
| Concentration | Particles | Particles 10 nm | Nitrate |
| Control 0 ppm (mg/L) | 6256 | 7037 | 6731 |
| 0.25 ppm (mg/L) | 65 | 6908 | 80 |
| 0.5 ppm (mg/L) | 72 | 5416 | 75 |
| 1.0 ppm (mg/L) | 30 | 7189 | 84 |

TABLE 2
RLU Counts at 24 Hours Post Exposure to Nanoparticle Treatment

| | Attostat 8 nm | NIST Standard | AgNO3 Silver |
|---|---|---|---|
| Concentration | Particles | Particles 10 nm | Nitrate |
| Control 0 ppm (mg/L) | 7595 | 5421 | 7342 |
| 0.25 ppm (mg/L) | 25 | 5691 | 25 |
| 0.5 ppm (mg/L) | 8 | 3950 | 46 |
| 1.0 ppm (mg/L) | 30 | 3834 | 30 |

Tables 3 and 4 represent the data in terms of comparing each treatment to its respective control at 12 and 24 hours post treatment, respectively.

TABLE 3
RLU as percentage of control at 12 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 1.0% | 98.2% | 1.2% |
| 0.5 ppm (mg/L) | 1.1% | 77.0% | 1.1% |
| 1.0 ppm (mg/L) | 0.6% | 102.2% | 1.3% |

TABLE 4
RLU as percentage of control at 24 Hours Post Treatment

| | Attostat 8 nm | NIST Standard | AgNO3 Silver |
|---|---|---|---|
| Concentration | Particles | Particles 10 nm | Nitrate |
| Control 0 ppm (mg/L) | 100% | 100% | 100% |
| 0.25 ppm (mg/L) | 0.33% | 105% | 0.34% |
| 0.5 ppm (mg/L) | 0.11% | 72.9% | 0.62% |
| 1.0 ppm (mg/L) | 0.39% | 70.7% | 0.41% |

As shown, at all concentrations tested, the Attostat nanoparticles reduced the number of RLU counts to less than 1.5% from the control baseline at both the 12 hour and 24 hour measurement periods. Anything below 1.5% is below level of accurate detection and is considered a complete kill.

The Attostat nanoparticles effectively reduced RLU counts to below the 1.5% threshold at all tested concentrations. The NIST nanoparticles appeared to show a trend toward greater efficacy at higher concentrations, which would correspond to a normal diffusion model, but even at the highest tested concentration still only reached an RLU count of 70.7% of the initial control baseline at the 24 hour measurement.

The low antimicrobial efficacy of the NIST nanoparticles at the concentrations tested as compared to the silver nitrate could potentially be explained by the lower conductivity, and thus lower ion concentration, of the NIST nanoparticles as compared to the silver nitrate. However, the significant efficacy of the Attostat nanoparticles was surprising given the fact that the Attostat nanoparticles have significantly low to non-detectable levels of ions, even lower than the NIST particles. The Attostat nanoparticles continued to provide antimicrobial activity through the 24-hour testing period with no signs of reduced efficacy.

The foregoing test results show a trend of comparative efficacy at increasing concentrations that would reasonably be expected to continue at even higher concentrations, such as at 5 ppm, 10 ppm, 15 ppm, 20 ppm, 30 ppm, 40 ppm, 50 ppm, 75 ppm, and 100 ppm (i.e., in which the Attostat 8 nm nanoparticles would have significantly lower RLU counts at increasing concentrations compared to the NIST Standard nanoparticles 10 nm), although the difference may narrow as the concentration is increased.

### Example 6

An assay for antiviral activity is designed to test and compare the antiviral activity of nanoparticle compositions of different sizes. All tested nanoparticle compositions are spherical, metal nanoparticles formed via laser ablation as described herein. A first test composition includes nanoparticles with a mean diameter of 4 nm. A second test composition includes nanoparticles with a mean diameter of 8 nm. A third test composition includes nanoparticles with a mean diameter of 14 nm. All reagents, test microbes, concentrations, treatment duration, and other test protocols are held the same across tests. The first test composition, with mean particle diameter of 4 nm, has greater antiviral activity than the second test composition (mean particle diameter 8 nm) or the third test composition (mean particle diameter 14 nm).

### Example 7

An assay for antibacterial activity is designed to test and compare the antibacterial activity of nanoparticle compositions of different sizes. All tested nanoparticle compositions are spherical, metal nanoparticles formed via laser ablation as described herein. A first test composition includes nanoparticles with a mean diameter of 4 nm. A second test composition includes nanoparticles with a mean diameter of 8 nm. A third test composition includes nanoparticles with a mean diameter of 14 nm. All reagents, test microbes, concentrations, treatment duration, and other test protocols are held the same across tests. The second test composition, with mean particle diameter of 8 nm, has greater antibacterial activity than the first test composition (mean particle diameter 4 nm) or the third test composition (mean particle diameter 14 nm).

### Example 8

An assay for antifungal activity is designed to test and compare the antifungal activity of nanoparticle compositions of different sizes. All tested nanoparticle compositions are spherical, metal nanoparticles formed via laser ablation as described herein. A first test composition includes nanoparticles with a mean diameter of 4 nm. A second test composition includes nanoparticles with a mean diameter of 8 nm. A third test composition includes nanoparticles with a mean diameter of 14 nm. All reagents, test microbes, concentrations, treatment duration, and other test protocols are held the same across tests. The third test composition, with mean particle diameter of 14 nm, has greater antifungal activity than the first test composition (mean particle diameter 4 nm) or the second test composition (mean particle diameter 8 nm).

### Example 9

Nonionic spherical silver nanoparticles having a mean particle size in a range of about 8 nm to about 10 nm, in which at least 99% of the silver nanoparticles have a particle size that is within 1 nm of the mean diameter, are highly effective in targeting and killing bacteria. They are significantly more effective at killing bacteria at lower concentrations compared to silver nanoparticles having greater size variability at a given concentration. They are also significantly more effective at killing bacteria than either viruses or fungi at a given concentration.

### Example 10

Nonionic spherical silver nanoparticles having a mean particle size in a range of about 3 nm to about 6 nm, in which at least 99% of the silver nanoparticles have a particle size that is within ±1 nm of the mean diameter, are highly effective in targeting and killing viruses. They are significantly more effective at killing viruses at lower concentrations compared to silver nanoparticles having greater size variability at a given concentration. They are also significantly more effective at killing viruses than either bacteria or fungi at a given concentration.

### Example 11

Nonionic spherical silver nanoparticles having a mean particle size in a range of about 12 nm to about 15 nm, in which at least 99% of the silver nanoparticles have a particle size that is within ±1 nm of the mean diameter, are effective in targeting and killing fungi. They are significantly more effective at killing fungi at lower concentrations compared to silver nanoparticles having greater size variability at a given concentration. They are also significantly more effective at killing fungi than either viruses or bacteria at a given concentration.

### Example 12

An antibacterial efficacy test is carried out comparing a “Niedermeyer” nanoparticle formulation (8 nm size) against silver nitrate and against the National Institute of Standards and Technology (NIST) Standard Nanocomposix 10 nm silver nanoparticles (see the nanoparticles tested in Example 5). The NIST nanoparticles are formed by a chemical reduction process that utilizes citrate as reducing and capping agent. The NIST nanoparticles have a conductivity similar to the “Meso” nanoparticles of Example 4, with detectable but low levels of silver ions.

Relative Light Unit (RLU) counts are recorded at 12 hours and 24 hours post treatment. RLU measurements are carried out using a Hygiena SystemSURE Plus V.2 SN067503 RLU meter with Hygenia AquaSnap TOTAL ATP Water Test Cat #U143 Lot #153019. Culturing media is Hardy Diagnostics Buffered Peptone Water Lot #118272. Samples are prepared with the nanoparticle treatments and then diluted with the media to provide the tested concentrations. The test organism(s) (such as but not limited to Microbiologics, E. coli, KwikStik, ATCC #51813, Ref #0791 K, Lot #791-1-6) are incubated in fresh Buffered Peptone Water growth media for 24 hours prior to exposure to the nanoparticle treatments. Table 5 illustrates the results of RLU counts 12 hours or more post-nanoparticle treatment.

TABLE 5
RLU as percentage of control at 12 Hours Post Treatment

| | Attostat 8 nm | NIST Standard | AgNO3 Silver |
|---|---|---|---|
| Concentration | Particles | Particles 10nm | Nitrate |
| Control 0 ppm (mg/L) | 100% | 100% | 100% |
| 0.25 ppm (mg/L) | 1.0% | 98.2% | 1.2% |
| 0.5 ppm (mg/L) | 1.1% | 77.0% | 1.1% |
| 1.0 ppm (mg/L) | 0.6% | 102.2% | 1.3% |
| 5.0 ppm (mg/L) | 0.5% | 73.3% | 1.1% |
| 10.0 ppm (mg/L) | 0.5% | 64.8% | 1.0% |
| 20.0 ppm (mg/L) | 0.5% | 45.4% | 0.8% |
| 50.0 ppm (mg/L) | 0.4% | 22.1% | 0.8% |
| 80.0 ppm (mg/L) | 0.3% | 10.4% | 0.9% |
| 100.0 ppm (mg/L) | 0.3% | 1.5% | 0.6% |
| 200.0 ppm (mg/L) | 0.3% | 1.3% | 0.3% |
| 300.0 ppm (mg/L) | 0.2% | 1.2% | 0.3% |
| 600.0 ppm (mg/L) | 0.1% | 1.2% | 0.1% |

As shown, at all concentrations, the Attostat 8 nm nanoparticles reduce the number of RLU counts to less than 1.5% from the control baseline at the 12-hour measurement periods. Anything below 1.5% is below level of accurate detection and is considered a complete kill.

The Attostat 8 nm nanoparticles effectively reduce RLU counts to below the 1.5% threshold at all tested concentrations. The NIST 10 nm nanoparticles show a trend toward greater efficacy at higher concentrations, which corresponds to a normal diffusion model. For example, as seen at 100.0 ppm, the NIST nanoparticles reach an RLU count of 1.5%, which is indicative of almost a complete kill. However, additional testing indicates that, although the NIST nanoparticles achieve comparable antimicrobial effectiveness at substantially higher concentrations, such activity is not persistent, as effectiveness is not maintained across lower concentrations or repeated exposures (e.g., 28 cycles). In contrast, the Attostat 8 nm nanoparticles maintain consistent antimicrobial activity across all tested concentrations and at repeated exposures (e.g., 28 cycles), further demonstrating a persistent antimicrobial effect.

The antimicrobial compositions described herein exhibit persistent antimicrobial activity, defined as the ability to maintain antimicrobial effectiveness over extended periods of time and repeated exposure conditions (e.g., 28 cycles) without significant loss of efficacy (i.e., because the disclosed antimicrobial compositions utilize silver nanoparticles that kills without significant release of silver ions, which means they do not become depleted over time like conventional colloidal silver ions). Such persistence may be characterized by sustained suppression or elimination of microbial populations following initial treatment, even in the absence of reapplication or increasing concentrations. In contrast to conventional antimicrobial agents, including ionic silver and chemically synthesized silver nanoparticles, which may exhibit diminished effectiveness over time due to depletion, aggregation, or the development of microbial tolerance or resistance, the compositions described herein maintain consistent antimicrobial performance across multiple exposure cycles. Thus, the antimicrobial compositions disclosed herein can advantageously exhibit persistent antimicrobial activity over multiple uses or extended use periods.

### Example 13

A study is conducted to evaluate long-term antimicrobial persistence and durability of an EVQ-218 nanoparticle formulation relative to commercially available nanoComposix colloidal silver nanoparticles (NIST). The testing is designed to determine whether antimicrobial efficacy is maintained over an extended period, including up to 30 days, under conditions involving repeated bacterial challenge. In particular, the study assesses the ability of each formulation to retain antimicrobial activity without substantial loss of efficacy over multiple sequential exposures to microorganisms.

Unlike conventional minimum inhibitory concentration (MIC) assays, which are subject to progressive dilution of the antimicrobial agent during repeated inoculation cycles, the present study is structured to maintain a substantially constant concentration of the test compositions throughout the duration of the experiment. Dilution effects are minimized by reintroducing bacterial inoculum in relatively small volumes, thereby enabling direct evaluation of persistence, reuse potential, and resistance to activity decay over time.

Antimicrobial activity is evaluated against Escherichia co/i. Test samples are prepared by introducing EVQ-218 and NIST nanoparticles into tryptic soy broth at a concentration of approximately 100 μg/mL. Samples are aliquoted into sterile vessels in replicate, and appropriate controls are included, including untreated controls and sterility controls.

A bacterial inoculum is prepared from a log-phase culture and introduced into each sample at an initial concentration of approximately 10⁶ CFU/mL. Following inoculation, samples are incubated at 37 °C for approximately 24 hours.

After the initial incubation period and at subsequent daily time points, aliquots are removed from each sample and analyzed for microbial survival by plating on agar media and determining colony forming units (CFU) or by qualitative assessment of growth. Following each assessment, the samples are rechallenged with fresh bacterial inoculum while maintaining substantially constant overall sample volume and antimicrobial concentration. This process is repeated over multiple cycles, including daily rechallenge over a period of up to 30 days.

The results illustrate antimicrobial performance as a function of time, including measures of microbial growth, time to onset of detectable regrowth, and observable changes in the test compositions. Sustained suppression of microbial growth over repeated challenge cycles indicates enhanced persistence and long-term antimicrobial efficacy, whereas earlier regrowth indicates less persistent efficacy. The EVQ-218 nanoparticles demonstrate greater persistent efficacy than the NIST nanoparticles at the same concentration.

### Example 14

Pseudomonas aeruginosa (ATCC 15442) was grown in cationic Mueller Hinton broth (CMHB) in several wells of a 96 well plate. A 96-peg lid was placed on the plate to bring the pegs into contact with respective wells. See, e.g., T. Tsukatani, F. Sakata, R. Kuroda, “A rapid and simple measurement method for biofilm formation inhibitory activity using 96-pin microtiter plate lids”, World J. Microbiol. Biotechnol., 2020, doi:10.1007/s 11274-020-02964 6.

After a first growth period of 20 hours (temperature=34 °C) to induce creation of biofilms, the peg lid was removed and rinsed three times with a wash solution, leaving biofilms on the peg surfaces. The 96-peg lid was then transferred to a 96-well plate with fresh CNM media. Different wells also included different treatments, each provided at a concentration of 4 ppm:
EVQ-218: spherical, nonionic silver nanoparticles formed by laser ablation and that are uncoated (no capping agents required for manufacture).
SB: a colloidal silver product (SB22299) available from Silver Biotics (American Fork, UT), determined to be silver nanoparticles formed from a process using a silver electrode in a reducing/capping salt, with average size of approximately 10 nm though mostly present in ~50 nm agglomerates.
NIST: standard nanoparticle composition for NIST, available from nanoComposix (San Diego, CA); formed through a chemical reduction process with reducing/capping agents and known to release silver ions; average size of approximately 10 nm, but with a broader particle size distribution.
AZ: Azithromycin, used as a positive control.

The pegs were contacted with the nanoparticle treatments for a treatment period of 20 hours, after which the 96-peg lid was removed, rinsed three times with a wash solution, and transferred to a 96-well plate containing fresh CMHB media. Growth in the media of the wells was monitored with a plate spectrometry reader (TECAN F200) at 595 nm. Higher absorbance in the measured media is indicative of greater bacterial survival within the biofilms, whereas lower absorbance is indicative of greater efficacy of the treatment (i.e., greater ability of the nanoparticle treatment to penetrate the biofilm and inhibit the bacteria).

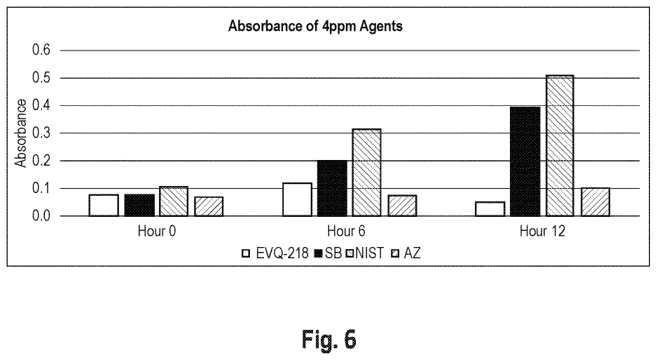

Results are illustrated in FIG. 6. As shown, the EVQ-218 nanoparticles were able to prevent increases in absorbance across the 12 hour measurement period, on par with the azithromycin positive control, indicating that the EVQ-218 nanoparticles were able to effectively penetrate the biofilm and inhibit the bacteria within the film. The comparison nanoparticle treatments (SB and NIST) did not show such efficacy, and were instead associated with an increase in bacterial growth in the media, indicating that these treatments did not significantly penetrate the formed biofilms.

This surprising difference in efficacy illustrates that nonionic, spherical-shaped silver nanoparticles that are uncoated and do not operate through the release of silver ions can indeed effectively penetrate biofilms to kill bacteria within and underneath the films, whereas the other types of nanoparticles failed to effectively prevent re-inoculation of media by the biofilms.

### Example 15

The silver EVQ-218 nanoparticles used in Example 14 were subjected to serial passage testing and did not show signs of generating resistance even after 28 passages.

The serial passage testing was independently carried out on Pseudomonas aeruginosa (ATCC 15442) and Escherichia coli (ATCC 25922), each at two silver nanoparticle concentrations (4.75 ppm and 2 ppm). The bacteria were streaked onto tryptic soy agar (TSA) plates and incubated overnight at 37 °C. Subsequently, 10 ml containers with Mueller Hinton broth and silver nanoparticle solution (silver broth mixture) were inoculated, with final concentration of silver nanoparticles set to 4.75 ppm or 2 ppm. The containers were incubated at 37 °C and 250 RPM for 24 to 36 hours and monitored for growth, after which serial passage was performed by inoculating fresh silver broth mixture. Every 5-7 days, the most recent cultures were streaked onto fresh TSA plates to determine whether resistance had been generated. No measurable resistance was determined even after 28 passages.

Additional antibiotic resistance testing is described in “Escaping the ESKAPE Antimicrobial Resistant Cycle with EVQ-218,” by Sharif et al. (EVOQ Nano, Inc., manuscript version dated Feb. 4, 2026) (https://doi.org/10.3390/1010000) (herein referred to as “Sharif et al.”), which is incorporated herein by reference. Briefly, Sharif et al. describes EVQ-218 silver nanoparticles evaluated against World Health Organization (WHO)-designated ESKAPE pathogens (Enterococcus faecium, Staphylococcus aureus, Klebsiella pneumoniae, Acinetobacter baumannii, Pseudomonas aeruginosa, and Enterobacter spp.), six of the most critical antibiotic-resistant pathogens. Across extended serial passage experiments performed under both sub- and supra-minimum inhibitory concentration (MIC) conditions, EVQ-218 did not exhibit measurable increases in MIC (<15 μgmL-1) or phenotypic indicators of adaptive resistance. All time-kill assays demonstrated complete bacterial killing within 30 minutes at concentrations ≤5 μgmL-1, with the exception of the Gram-positive Staphylococcus aureus, which required 90 minutes to achieve comparable killing. In contrast, parallel exposure to tobramycin and ciprofloxacin resulted in rapid and sustained MIC elevation. Notably, isolates that acquired resistance to either comparator antibiotic retained susceptibility to EVQ-218, demonstrating a lack of cross-resistance.

The studies described in Sharif et al. further demonstrated that EVQ-218 maintains antimicrobial efficacy under both constant and increasing exposure conditions. In particular, serial passage experiments conducted across multiple bacterial generations, including under conditions in which the concentration of EVQ-218 was progressively increased, showed no upward shift in minimum inhibitory concentration (MIC) values, indicating an absence of adaptive resistance or tolerance development.

The antimicrobial activity of EVQ-218 was further characterized as concentration-dependent and rate-dependent, with increased concentrations producing more rapid and complete bacterial killing. At concentrations at or above the MIC, rapid bactericidal effects were observed, while at sub-inhibitory concentrations, growth suppression was maintained without evidence of recovery or adaptation over time. This concentration-dependent behavior supports effectiveness across a range of concentrations, including both lower concentrations providing sustained antimicrobial suppression and higher concentrations providing rapid bactericidal activity.

The testing in Sharif et al. further demonstrated broad-spectrum antimicrobial activity across both genetically diverse Gram-positive and Gram-negative pathogen species, including clinically relevant and multidrug-resistant pathogens. For example, and without limitation, Gram-positive bacteria such as Staphylococcus species (spp.), Enterococcus spp., Streptococcus spp., Bacillus spp., and Clostridium spp., and Gram-negative bacteria such as Escherichia spp., Pseudomonas spp., Klebsiella spp., Acinetobacter spp., Enterobacter spp., Salmonella spp., Shigella spp., Proteus spp., Serratia spp., Burkholderia spp., and Stenotrophomonas spp., including antibiotic-resistant and multidrug-resistant strains thereof. In addition, isolates that developed resistance to conventional antibiotics, including aminoglycosides and fluoroquinolones, remained susceptible to EVQ-218, demonstrating a lack of cross-resistance and further evidencing a mechanism of action distinct from conventional antimicrobial agents.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of killing or deactivating a target microbe, comprising:
providing an antimicrobial composition that kills or deactivates a bacteria, the antimicrobial composition comprising:
a carrier; and
a plurality of nonionic, ground state spherical-shaped silver nanoparticles formed by laser ablation from silver or a silver alloy and having a mean diameter in a range of about 7 nm to about 12 nm so as to kill or deactivate the selected bacteria, wherein at least 99% of the spherical-shaped, nonionic metal nanoparticles have a diameter within ±3 nm of the mean diameter, wherein the spherical-shaped silver nanoparticles are present in the antimicrobial composition at a concentration of at least 0.25 ppm;
applying the antimicrobial composition to the bacteria or to a substrate containing the bacteria; and
the metal nanoparticles killing or deactivating the bacteria with greater efficacy than chemically synthesized colloidal silver nanoparticles at the same concentration.

2. The method of claim 1, wherein the spherical-shaped silver nanoparticles kill or deactivate the bacteria with greater persistent efficacy than chemically synthesized colloidal silver nanoparticles at the same concentration.

3. The method of claim 1, wherein the spherical-shaped silver nanoparticles have a mean diameter in a range of about 8 nm to about 10 nm.

4. The method of claim 1, wherein the spherical-shaped silver nanoparticles comprise silver or an alloy of silver and gold.

5. The method of claim 1, wherein the carrier is a liquid in which the metal nanoparticles are dispersed.

6. The method of claim 1, wherein the substrate is a non-living object.

7. The method of claim 1, wherein the substrate is a living organism.

8. The method of claim 1, wherein the spherical-shaped silver nanoparticles have a concentration of about 0.5 ppm to about 100 ppm.

9. The method of claim 1, wherein the spherical-shaped silver nanoparticles have a concentration of about 1 ppm to about 50 ppm.

10. A method of killing or deactivating a target microbe, comprising:
providing an antimicrobial composition that kills or deactivates a bacterium, the antimicrobial composition comprising:
a carrier; and
a plurality of nonionic, ground state spherical-shaped silver nanoparticles formed from silver or a silver alloy by laser ablation and having a mean diameter in a range of about 8 nm to about 10 nm so as to kill or deactivate the selected bacterium, wherein at least 99% of the spherical-shaped, nonionic metal nanoparticles have a diameter within ±3 nm of the mean diameter, wherein the spherical-shaped, nonionic metal nanoparticles are present in the antimicrobial composition in a concentration of about 0.25 ppm to about 100 ppm;
applying the antimicrobial composition to the bacterium or to a substrate containing the bacterium; and
the metal nanoparticles killing or deactivating the bacterium with greater efficacy than chemically synthesized colloidal silver nanoparticles at the same concentration.

11. The method of claim 10, wherein the spherical-shaped, nonionic metal nanoparticles comprise silver or an alloy of silver and gold.

12. The method of claim 10, wherein the carrier is a liquid in which the metal nanoparticles are colloidally dispersed.

13. The method of claim 10, wherein the substrate is a non-living object.

14. The method of claim 10, wherein the substrate is a living organism.

15. The method of claim 10, wherein the spherical-shaped silver nanoparticles have a concentration of about 0.5 ppm to about 50 ppm.

16. An antimicrobial composition comprising:
a carrier; and
a plurality of nonionic, ground state spherical-shaped silver nanoparticles formed by laser ablation from silver or a silver alloy and having a mean diameter in a range of 7 nm to 12 nm to kill or deactivate antibiotic resistant bacteria, wherein at least 99% of the spherical-shaped, nonionic metal nanoparticles have a diameter within 3 nm of the mean diameter, wherein the spherical-shaped silver nanoparticles are present in the antimicrobial composition in a concentration of at least 0.25 ppm;
wherein the antimicrobial composition is effective in killing antibiotic-resistant bacteria with greater efficacy than chemically synthesized colloidal silver nanoparticles at the same concentration and does not induce an increase in resistance in the bacteria upon repeated exposure.

17. The antimicrobial composition of claim 16, wherein the spherical-shaped silver nanoparticles have a mean diameter in a range of about 8 nm to about 10 nm.

18. The antimicrobial composition of claim 16, wherein the spherical-shaped silver nanoparticles have a concentration of about 0.5 ppm to about 50 ppm.

19. The antimicrobial composition of claim 16, wherein the spherical-shaped silver nanoparticles of about 0.25 ppm to about 100 ppm.

20. The antimicrobial composition of claim 16, wherein the antimicrobial composition is effective against both gram negative and gram positive bacteria.
